(19) United States
(12) Patent Application Publication
(10) Pub. No.: US 2010/0174202 A1
(43) Pub. Date: Jul. 8, 2010

(54) METHOD AND SYSTEM FOR COMBINING OSCILLOMETRIC BLOOD PRESSURE ENVELOPE DATA OBTAINED FROM DIFFERENT SIGNAL PROCESSING PATHS

(75) Inventors: Lawrence T. Hersh, Milwaukee, WI (US); Sai Kolluri, Mequon, WI (US); Bruce A. Friedman, Jasper, GA (US); Richard Medero, Tampa, FL (US)

Correspondence Address: Andrus, Sceales, Starke & Sawall, LLP, 100 East Wisconsin Avenue, Suite 1100, Milwaukee, WI, 53202-4178, US

(73) Assignee: THE GENERAL ELECTRIC COMPANY, Schenectady, NY (US)

(21) Appl. No.: 12/350,412

(22) Filed: Jan. 8, 2009

(51) Int. Cl. A61B 5/0225

(52) U.S. Cl. 600/494

(57) ABSTRACT

A blood pressure measurement system that utilizes a non-invasive blood pressure (NIBP) monitor having a blood pressure cuff and pressure transducer. The measurement system provides a plurality of separate processing techniques that each receive a plurality of oscillometric waveform sample values generated using the pressure transducer. Each of the processing techniques separately generates a set of envelope points based upon the oscillometric data values. The sets of envelope points are appropriately scaled such that the sets of scaled envelope points are combined with each other to create a set of combined, scaled envelope points. Various different methods can be used to scale the sets of envelope points prior to the combination of the scaled envelope points. Based upon the combination of scaled envelope points, the blood pressure is calculated and displayed by the NIBP monitor.

### BACKGROUND OF THE INVENTION

The present disclosure generally relates to automated blood pressure monitoring. More specifically, the present disclosure relates to automated blood pressure monitors that utilize multiple data processing techniques to process oscillometric data values obtained from a patient to generate multiple sets of data points that are combined to create a blood pressure measurement.

Non-invasive automated blood pressure monitors employ an inflatable cuff to exert controlled counter-pressure on the vasculature of a patient. One large class of such monitors, exemplified by that described in U.S. Pat. Nos. 4,349,034 and 4,360,029, both to Maynard Ramsey, III and commonly assigned herewith and incorporated by reference, employs the oscillometric methodology.

In accordance with the Ramsey patents, an inflatable cuff is suitably located on the limb of a patient and is pumped up to a predetermined pressure above the systolic pressure. The cuff pressure is then reduced in predetermined decrements, and at each level, pressure fluctuations are monitored. The resultant cuff pressure signal typically consists of a DC voltage with a small superimposed variational component caused by arterial blood pressure pulsations (referred to herein as “oscillation complexes” or just simply “oscillations”).

After suitable filtering to reject the DC component and amplification, peak amplitudes of the oscillations above a given base-line are measured and stored. As the cuff pressure decrementing continues, the peak amplitudes will normally increase from a lower level to a relative maximum, and thereafter will decrease. These amplitudes form an oscillometric envelope for the patient. The lowest cuff pressure at which the oscillations have a maximum value has been found to be representative of the mean arterial pressure (MAP) of the patient. Systolic and diastolic pressures can be derived either as predetermined fractions of the oscillation size at MAP, or by more sophisticated methods of processing of the oscillation complexes.

The step deflation technique as set forth in the Ramsey patents is the commercial standard of operation. A large percentage of clinically acceptable automated blood pressure monitors utilize the step deflation rationale. When in use, the blood pressure cuff is placed on the patient and the operator usually sets a time interval, typically from 1 to 90 minutes, at which blood pressure measurements are to be repeatedly made. The noninvasive blood pressure (NIBP) monitor automatically starts a blood pressure determination at the end of the set time interval. Alternatively, the operator may place the monitor in a mode for which each blood pressure determination is initiated by request. In either case, once the blood pressure determination is begun, the process is automatically controlled until the oscillometric information is obtained and blood pressure estimates are output.

FIG. 1 illustrates a simplified version of the oscillometric blood pressure monitor described in the aforementioned Ramsey patents. In FIG. 1, the arm 100 of a human subject is shown wearing a conventional flexible inflatable and deflatable cuff 101 for occluding the brachial artery when fully inflated. As the cuff 101 is deflated using deflate valve 102 having exhaust 103, the arterial occlusion is gradually relieved. The deflation of cuff 101 via deflate valve 102 is controlled by central processor 107 via control line 108.

A pressure transducer 104 is coupled by a duct 105 to the cuff 101 for sensing the pressure therein. In accordance with conventional oscillometric techniques, pressure oscillations in the artery create small pressure changes in the cuff 101, and these pressure oscillations are converted into an electrical signal by transducer 104 and coupled over path 106 to the central processor 107 for processing. In addition, a source of pressurized air 109 is connected via a duct 110 through an inflate valve 111 and a duct 112 to the pressure cuff 101. The inflate valve 111 is electrically controlled through a connection 113 from the central processor 107. Also, the deflate valve 102 is connected by duct 114 via a branch connection 115 with the duct 112 leading to cuff 101.

During operation of the apparatus illustrated in FIG. 1, air under pressure at about 8-10 p.s.i. is typically available as the source of pressurized air 109. When it is desired to initiate a determination of blood pressure, the central processor 107 furnishes a signal over path 113 to open the inflate valve 111 after the deflate valve 102 is closed. Air from the source 109 is communicated through inflate valve 111 and duct 112 to inflate the cuff 101 to a desired level, preferably above the estimated systolic pressure of the patient. Central processor 107 responds to a signal on path 106 from the pressure transducer 104, which is indicative of the instantaneous pressure in the cuff 101, to interrupt the inflation of the cuff 101 when the pressure in the cuff 101 reaches a predetermined initial inflation pressure that is above the estimated systolic pressure of the patient. Such interruption is accomplished by sending a signal over path 113 instructing inflate valve 111 to close. Once inflate valve 111 has been closed, the blood pressure measurement can be obtained by commencing a deflate routine.

Actual measurement of the blood pressure under the control of the central processor 107 using the deflate valve 102 and the pressure transducer 104 can be accomplished in any suitable manner such as that disclosed in the aforementioned patents or as described below. At the completion of each measurement cycle, the deflate valve 102 can be re-opened long enough to relax the cuff pressure via exhaust 103.

Accordingly, when a blood pressure measurement is desired, the inflate valve 111 is opened while the cuff pressure is monitored using the pressure transducer 104 until the cuff pressure reaches the desired level. After reaching the desired pressure level, the inflate valve 111 is closed. Thereafter, the deflate valve 102 is operated using signal 108 from microprocessor 107 and the blood pressure measurement taken.

FIG. 2 illustrates a pressure versus time graph illustrating a conventional cuff step deflation and measurement cycle for a conventional NIBP monitor. As illustrated, the cuff is inflated to an initial inflation pressure 117 above the systolic pressure 119, and the cuff is then step deflated by a pressure step 121 to the next pressure level. A timeout duration is provided at each step during which the signal processing circuitry searches for oscillation complexes 122 including one or more oscillation pulses 123 in accordance with known techniques. At the end of timeout duration, the cuff pressure is decremented whether or not oscillation complexes have been found. Alternatively, once oscillation complexes have been found, and determined to be adequate for measurement, the cuff pressure is decremented. This process of decrementing the pressure and searching for oscillation complexes is repeated until systolic, MAP, and diastolic pressure values can be calculated from the oscillometric envelope 116. The entire blood pressure determination process is then repeated at intervals set by the user, some other predetermined interval, or manually.

As shown in FIG. 2, the patient's arterial blood pressure forms an oscillometric envelope 116 comprised of a set of oscillation pulses 123 that each have an amplitude 124, which will be referred to as an oscillometric data value, measured at the different cuff pressures. From the oscillometric envelope 116, systolic, MAP and diastolic blood pressures are typically calculated. However, as noted in the afore-mentioned patents, it is desired that all artifact data be rejected from the measured data so that oscillometric envelope 116 contains only the desired amplitude data values 124 and no artifacts, thereby improving the accuracy of the blood pressure determinations.

Generally, conventional NIBP monitors of the type described in the afore-mentioned patents use oscillation amplitude matching at each pressure level as one of the ways to discriminate good oscillations from artifacts. In particular, pairs of oscillations are compared at each pressure level to determine if they are similar in amplitude and similar in other attributes, such as shape, area under the oscillation waveform, slope, and the like. If the oscillations compare within predetermined limits, the average amplitude and cuff pressure are stored and the pressure cuff is deflated to the next pressure level for another oscillation measurement. However, if the oscillations do not compare favorably, the first oscillation is typically discarded and another fresh oscillation is obtained. The monitor, maintaining the same pressure step, uses this newly obtained oscillation to compare with the one that was previously stored. This process normally continues until two successive oscillations match or a time limit is exceeded.

As discussed above, non-invasive blood pressure algorithms provide a blood pressure value at the end of the determination, which is then displayed to a user. However, during some blood pressure determinations, it is difficult to get data of high enough quality to enable an accurate blood pressure output. As an example, data gathered for the calculation of blood pressure could be corrupted from motion artifacts caused by the patient or by vibrations caused during transport. In the presence of such motion artifacts, signal-processing techniques that are beneficial for handling one type of artifact may not be desirable or may even be detrimental for other types of artifacts. During the calculation of the blood pressure, it is difficult to determine which processing technique may be best. Therefore, it is desirable to utilize multiple processing techniques and then combine the processing results, resulting in an optimal blood pressure measurement.

### BRIEF DESCRIPTION OF THE INVENTION

The following describes a method for measuring and displaying the blood pressure of a patient utilizing a non-invasive blood pressure (NIBP) monitor that has an inflatable and deflatable blood pressure cuff and a pressure transducer. The method obtains a cuff pressure versus time waveform from a pressure transducer of the NIBP monitor. The oscillometric waveform is sampled and the sample values are provided to a central processor that is programmed to carry out various signal processing techniques using these waveform sample values for the purpose of calculating blood pressure.

The plurality of oscillometric waveform sample values are received in the central processor and the central processor is operated to carry out at least a first and a second processing technique on the same oscillometric waveform sample values. Each of the processing techniques constructs a set of oscillometric envelope points based upon the received oscillometric waveform sample values. In particular, the processing techniques may be differently configured adaptive filters where the relative gains of the techniques for generating the oscillometric envelope data is unknown or very complex to determine. Since each of the processing techniques is carried out in a different manner, the first and second set of envelope points are different and distinct from each other.

Once the first and second sets of envelope points have been developed for each of the processing techniques, the first and second sets of envelope points are combined to create a combined set of envelope points. However, since the first and second processing techniques are different from each other, the first and second sets of envelope points will typically have different amplitude ranges and therefore either one or both of the first and second sets of envelope points must be scaled before the envelope points can be combined.

In one embodiment, a combining method is utilized that scales the first set of envelope points based upon the maximum value of the first set of envelope points. The scaled first envelope points thus have a value between 0 and 1. In addition to scaling the first set of envelope points, the second set of envelope points is also scaled based upon the maximum value of the second envelope points. After scaling, the second set of envelope points thus range between 0 and 1. After the first and second set of envelope points are appropriately scaled, the first and second sets of scaled envelope points are combined with each other to create a combined set of envelope points that create a final oscillometric envelope. Based upon the combined oscillometric envelope, the blood pressure for the patient is calculated.

In a second embodiment, a scaling pressure step is selected. Preferably, both the plurality of first envelope points and the plurality of second envelope points have an envelope point at or near the selected scaling pressure step. Once the scaling pressure step has been determined, a step scale factor is determined for the plurality of second envelope points. The step scale factor is based upon the ratio of the amplitude of the oscillation of the first set of envelope points to the amplitude of the oscillation of the second set of envelope points at the chosen scaling step. Once the step scale factor is determined, the plurality of second envelope points is multiplied by the step scale factor. Once the plurality of second envelope points have been appropriately scaled, the first envelope points and the scaled second envelope points are combined to once again create a scaled oscillometric envelope. The scaled oscillometric envelope is then utilized to determine the blood pressure for the patient.

In a third embodiment, the relative gain factors for the first and second processing techniques may be exactly known from the design and construction of the algorithm techniques. Additionally, these gain factors may not change with the patient or with time. In this case, the second set of envelope points can be scaled using a known factor and subsequently the second set of envelope points can be combined with the first. Similarly, the combined envelope points can be utilized to estimate the blood pressure for the patient.

Various other types of scaling techniques can be utilized to scale the plurality of first and second envelope points prior to combination of the envelope points. The combination of the scaled first and second envelope points allow the envelope points to be combined prior to determining the blood pressure for the patient.

### BRIEF DESCRIPTION OF THE DRAWINGS

The drawings illustrate the best mode presently contemplated of carrying out the disclosure. In the drawings:

FIG. 1 is a high level diagram of a non-invasive blood pressure (NIBP) monitoring system;

FIG. 2 illustrates oscillometric data including step deflate and oscillation amplitudes derived using the NIBP monitoring system of FIG. 1;

FIG. 3a-3c illustrate sets of data points from both a first processing technique and a second processing technique, as well as a combination of the data points;

FIG. 4 is a high level flowchart showing the use of multiple processing channels to build both first and second oscillometric envelope points and a combination of the first and second envelope points to estimate the blood pressure for a patient;

FIGS. 5a-5c illustrate the first and second oscillometric envelope points and a combination of the oscillometric envelope points to construct a combined envelope;

FIGS. 6a-6c illustrate incomplete first and second oscillometric envelope points and a combination of the scaled envelope points to create a combined blood pressure envelope;

FIG. 7 is a flowchart showing the steps to scale both the first and second envelope points such that the envelope points can be combined to create a combined envelope;

FIG. 8 is a flowchart illustrating the steps to scale the second envelope points based upon an oscillometric data value obtained at a common pressure step to create a combined blood pressure envelope; and

FIG. 9 is a flowchart illustrating the steps to combine the first and second oscillometric envelope points based upon application of a curve fitting algorithm to both the first and second envelope points prior to the combination of the envelope points.

### DETAILED DESCRIPTION OF THE INVENTION

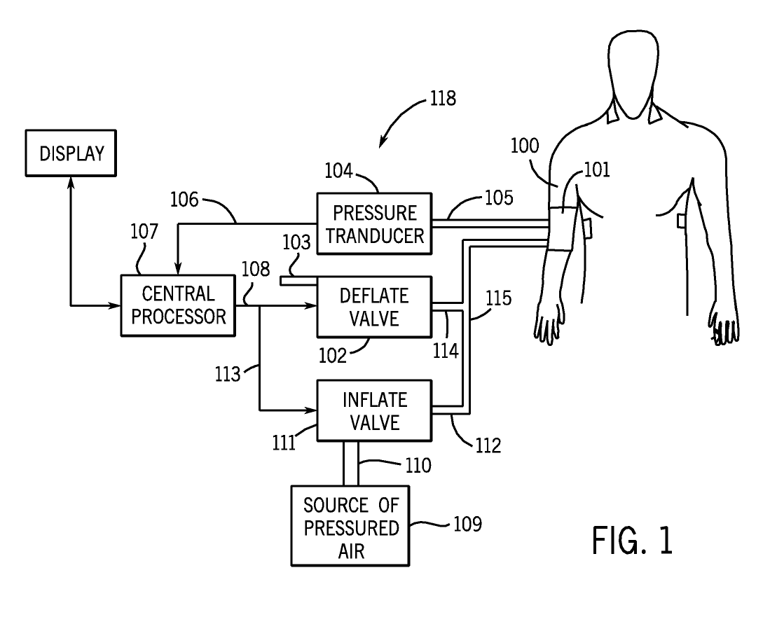

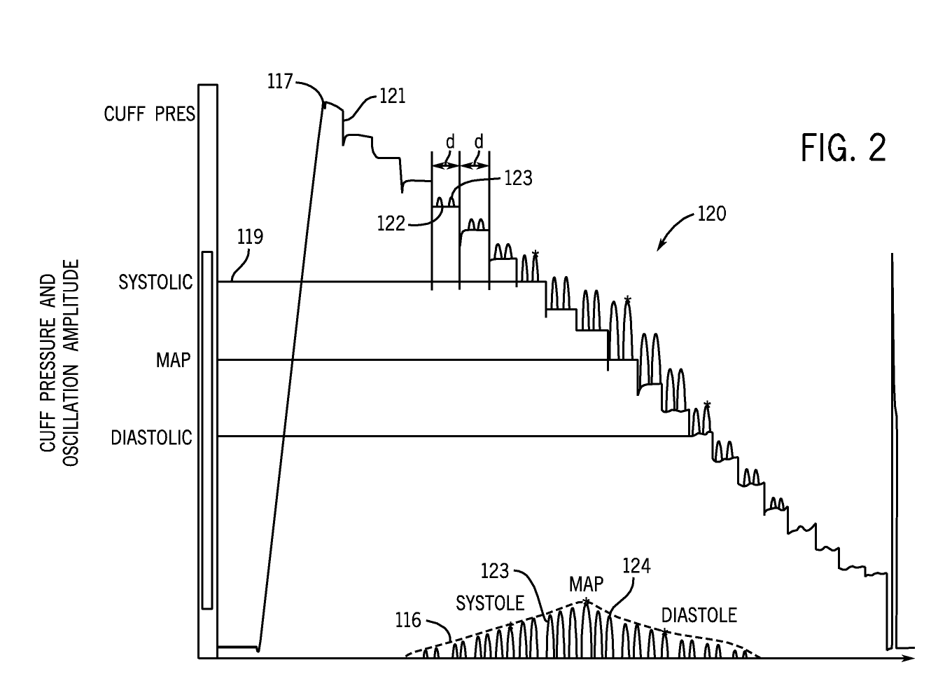

As described previously in the description of FIGS. 1 and 2, the NIBP monitoring system 118 generates a cuff pressure deflation profile 120 and obtains oscillometric envelope points 124 by processing cuff pressure waveform sample values corresponding to each pressure step 121 that generally fit close to a bell-shaped envelope 116, as shown in FIG. 2. In the measurement shown in FIG. 2, the oscillometric envelope 116 is created with high quality, clean data.

In a typical NIBP monitoring system, the cuff pressure waveform sample values are filtered using a conventional band pass filters having a lower cutoff frequency near 0.5 Hz and an upper cutoff frequency near 7.2 Hz. Although this band pass filter has proven to be an effective data processing technique for filtering out unwanted noise and artifacts, the band pass may sometimes be ineffective for removing artifacts due to patient motion or transportation.

As described previously, the pressure transducer 104 shown in FIG. 1 generates an oscillometric waveform to obtain envelope values for each cuff pressure step, as can be understood in FIGS. 1 and 2. The oscillometric waveform is fed to the central processor 107 along path 106 for sampling and further processing. The present disclosure provides for multiple methods of operating the central processor 107 to process the oscillometric waveform received from the pressure transducer 104.

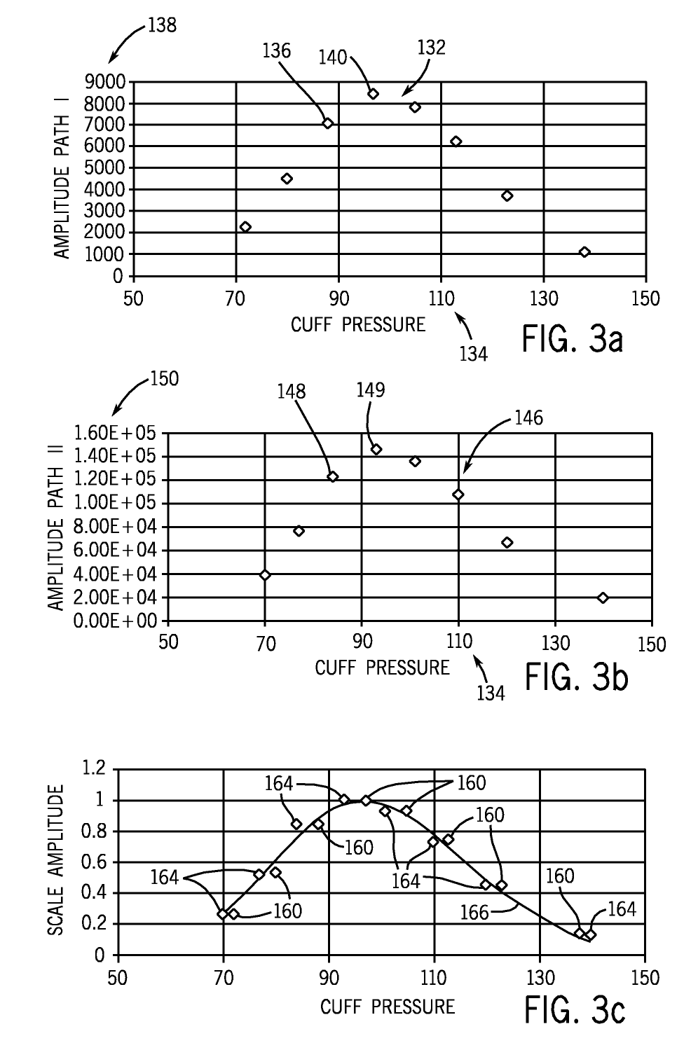

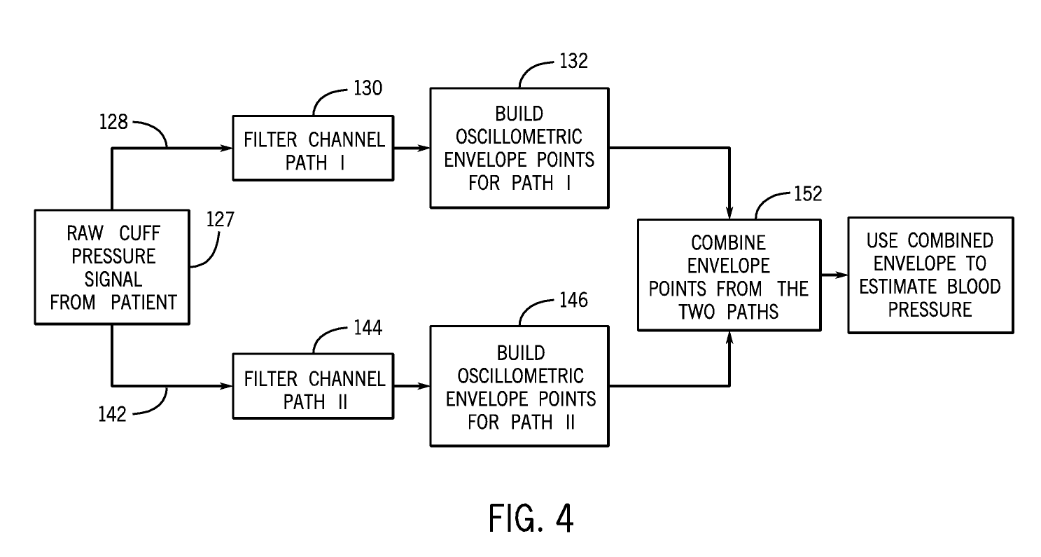

FIG. 4 generally illustrates the steps performed by the central processor 107 in calculating a blood pressure estimate for the patient. As illustrated in FIG. 4, the oscillometric waveforms are received from the patient in step 127. As described previously, the oscillometric waveforms are received from the pressure transducer and are used to find a series of oscillometric complex amplitude measurements determined at the various cuff pressures defined by the pressure steps 121 that form part of the deflation profile 120 shown in FIG. 2. As shown in FIG. 4, the oscillometric waveforms are fed through a first data processing channel 128 that leads to a first data filter 130. The first data filter 130 carries out a first processing technique and creates a first set of envelope points 132. As an example, the processing technique carried out by the first data filter 130 could be a bandpass filter having a mid-band within the usual physiological oscillometric frequency range. As an example, the pass band for the filter 130 may have a lower cutoff point near 0.5 Hz and upper cutoff point near 7.2 Hz. After the oscillometric data values are passed through the first data filter 130, the first data processing channel 128 can create an oscillometric envelope, such as shown in FIG. 3a.

As illustrated in FIG. 3a, the first set of envelope points 132 includes a plurality of individual envelope points 136 at various different cuff pressures 134. Each of the envelope points 136 relates to a cuff pressure 134 and amplitude 138. As illustrated in FIG. 3a, the first set of envelope points 132 creates a curve that includes maximum amplitude 140. In the first set of envelope points shown after filtering in FIG. 3a, the envelope points include very little noise, which results in the typical bell-shaped appearance shown in FIG. 3a.

Referring back to FIG. 4, the system further includes a second processing channel 142 that directs the oscillometric data values to a second data filter 144. The second data filter 144 performs a second processing technique on the oscillometric waveforms to construct a second set of envelope points 146. In the embodiment shown in FIG. 4, the second data filter 144 could be a low-band filter that may be selected to have pass band from 0.5 Hz to 3 Hz. Alternatively, the second data filter 144 could be a high-band filter that builds the second set of envelope points 146 utilizing a pass band from 3 Hz to 7.2 Hz. Although two different types of pass-band filters are described for the first data filter 130 and the second data filter 144, it should be understood that various different processing techniques could be utilized while operating within the scope of the present disclosure. Further, although two data filters 130, 144 are shown in the embodiment of FIG. 4, it should be understood that additional data processing channels could be utilized while operating within the scope of the present disclosure.

Further, in addition to the band pass filters shown in the processing channels 128, 142, the system may also include other data processing techniques to construct an oscillometric envelope. As an example, a frequency domain filter that processes the oscillometric data values could be utilized. This type of filter picks specific and multiple frequency components (magnitude and phase) to construct multiple envelopes as output. The output of the frequency domain filter could also be utilized to calculate the blood pressure for the patient.

In another alternate type of processing technique, the system could take advantage of the timing relationship of the oscillations with respect to ECG and SpO₂ or plethysmographic measurements. As an example, the ECG information could be used to control opening a window in time of a particular duration when the blood pressure oscillation is expected. In this way the oscillations would be obtained at the times when they were most likely to occur while disregarding artifacts that might be present outside of the time window.

Although various types of processing techniques are described, other processing techniques are also contemplated as being within the scope of the present disclosure. As an example, it is contemplated that the oscillometric envelopes can be calculated using adaptive filtering by configuring the filter properties based on the heart rate or peak match filtering and template matching. In any case, the processing technique of the data channel generates a set of envelope points, such as those shown by reference numerals 132 and 146.

Referring now to FIG. 3b, the second set of envelope points 146 are shown after the second processing technique. The second set of envelope points 146 are distributed over the same range of cuff pressure 134. However, the second set of individual envelope points 148 have a different range of amplitudes 150, as compared to the amplitudes 138 shown in FIG. 3a.

In the embodiment shown in FIG. 4, the two different data processing techniques carried out in the processing channels 128, 142 may each be better at processing the oscillometric waveforms at different pressure steps. In this case, the resulting first set of envelope data points may have different amplitude characteristics as compared to the second set of envelope points. However, it is desirable to use all of the data from both the first and second processing channels 128, 142 to estimate the blood pressure for the patient, if possible. As an example, data processed utilizing the first technique may have a significant variation due to extreme artifacts, but the second data processing technique may have less variation due to the same artifacts. The exact characteristics of this artifact variation corrupting the data from the first processing technique may be unknown. By combining the data from the two processing techniques, a better blood pressure estimate can be determined.

As can be understood by FIGS. 3a and 3b, the first set of envelope points 132 has a different amplitude range 138 as compared to the second set of envelope points 146, which has the amplitude range shown by reference numeral 150. Therefore, before the envelope points can be combined in step 152 of FIG. 4, a combining technique must be utilized to combine the data points.

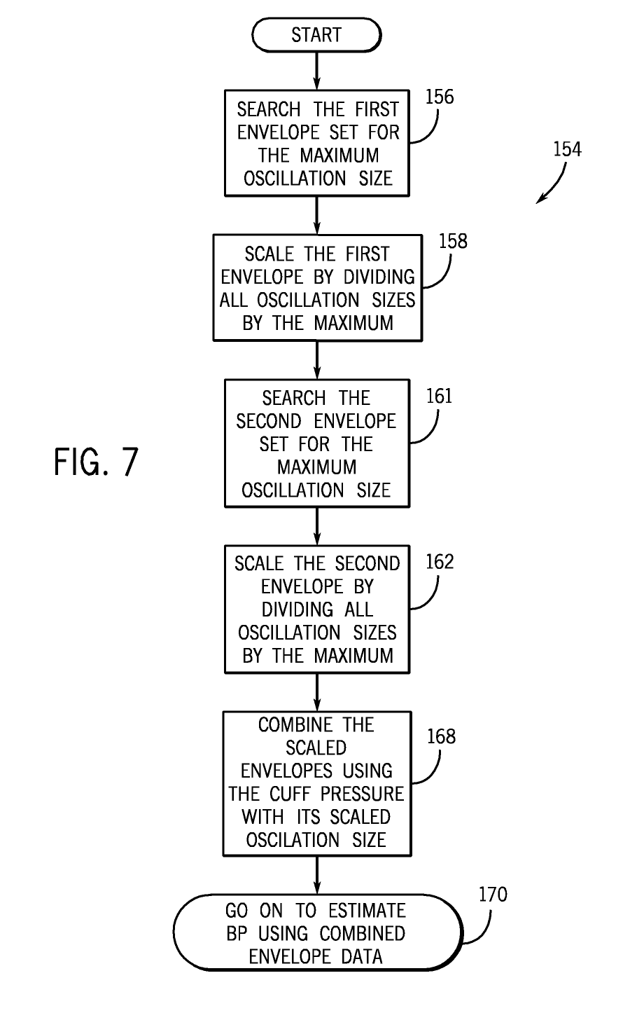

Referring now to FIG. 7, a first combining technique 154 is illustrated. Initially, the first combining technique searches the first set of envelope points shown in FIG. 3a for a maximum oscillation size, which is illustrated by reference numeral 140. After the combining technique finds the maximum oscillation size in step 156, the combining technique 154 scales all of the envelope points 136 by dividing each of the first envelope points 136 by the maximum amplitude, as shown in step 158. Since all of the first envelope points 136 are divided by the maximum amplitude, the scaled amplitude will be in the range of 0 to 1. The first set of scaled amplitude points are shown by reference characters 160 in FIG. 3c.

After the first set of envelope points 136 have been scaled, the processing technique 154 searches the second envelope set 146 for the maximum amplitude size 149 (FIG. 3b) from all of the second envelope points 148, as illustrated in step 161. Once the maximum amplitude 149 has been determined, each of the second envelope points 148 are scaled by dividing the second envelope points by the maximum, as shown in step 162.

Once the second set of envelope points have been scaled in accordance with step 162, the scaled second set of envelope points will have a scaled amplitude between 0 and 1. The second set of scaled amplitude points are generally shown in FIG. 3c by reference numeral 164. As illustrated in FIG. 3c, the combined first set of scaled envelope points 160 and the second set of scaled envelope points 164 create a combined, scaled oscillometric envelope 166. The combination of the scaled envelope in step 168 allows the combining technique 154 to utilize the results of both the first processing technique and the second processing technique by first scaling the results such that the first and second set of data points can be combined.

Referring back to FIG. 7, the combining technique 154 utilizes the combined envelope data shown in FIG. 3c to estimate the blood pressure of the patient utilizing conventional blood pressure estimating techniques, as shown in step 170.

In the embodiment shown in FIGS. 3a-3c, the first set of envelope points 132 and the second set of envelope points 146 provide a relatively clean and complete set of data points over the complete range of cuff pressure. The combination of the scaled envelope points shown in FIG. 3c results in an oscillometric envelope 166 that has a typical, appearance. Note that the processing for the first set of envelope points may determine envelope points that are at different cuff pressures than the processing for the second envelope points. This does not restrict the combination techniques described here.

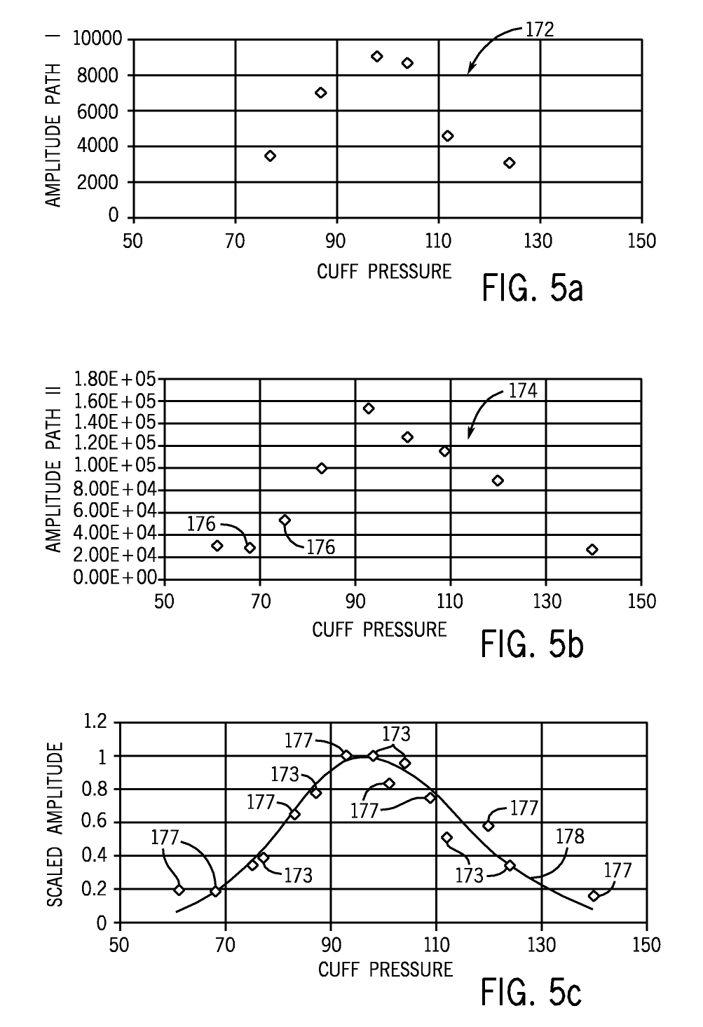

In the embodiment shown in FIGS. 5a-5c, the first set of envelope points 172 is incomplete as compared to FIG. 3a, while the second set of envelope points 174 includes inconsistent envelope points 176. Thus, if only the first set of envelope points 172 or the second set of envelope points 174 were utilized to estimate the blood pressure, the resulting blood pressure estimate may be incomplete or inaccurate.

In accordance with the present disclosure, the first combining technique 154 shown in FIG. 7 is applied to both the first set of envelope points 172 and the second set of envelope points 174, the combining technique creates the first set of scaled envelope points 173 and the second set of scaled envelope points 177 that are combined to create the resulting oscillometric envelope 178 shown in FIG. 5c. When the first set of envelope points 172 and the second set of envelope points 174 are scaled and combined, the scaled, combined envelope points 173 and 177 create a more typical oscillometric envelope 178, as shown in FIG. 5c.

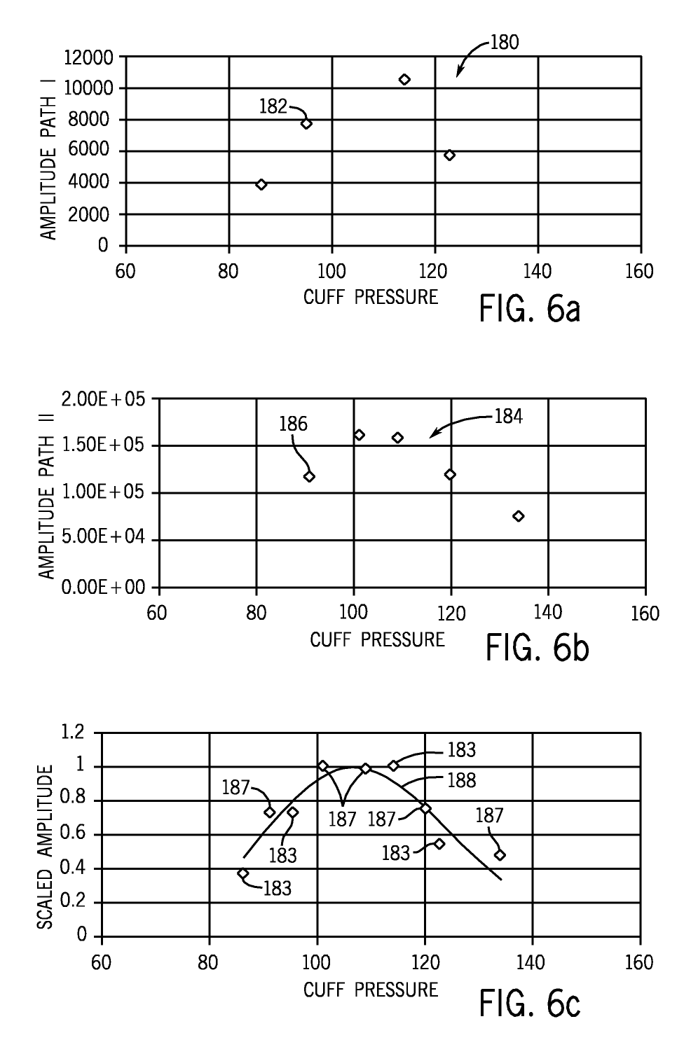

Referring now to FIGS. 6a-6c, the first set of envelope points 180 includes only four actual first envelope points 182. Likewise, the second set of envelope points 184 includes only five actual envelope points 186. Thus, neither the first set of envelope points 180 nor the second set of envelope points 184 are complete enough to create a reliable oscillometric envelope. In accordance with the present disclosure, the combining technique 154 shown in FIG. 7 results in a combination of the scaled first points 183 and the scaled second envelope points 187, as shown in FIG. 6c. The combined, scaled first and second envelope points create the oscillometric envelope 188, which is a combination of the scaled first set of envelope points and the scaled second set of envelope points. Thus, by utilizing the combining technique 154 of the present disclosure, the system utilizes the multiple processing techniques and combines the results of the processing techniques to create an oscillometric envelope.

Additionally, due to frequency content changes of the oscillometric complexes as the determination proceeds from pressure step to pressure step, the optimal filtering on the systolic side of the oscillometric envelope may be different from the optimal filtering used on the diastolic side of the envelope in order to best handle envelope construction. This means that the first processing technique may be better suited for systolic waveforms and the second processing technique may be better suited for diastolic waveforms. In this case, each processing technique will be used to provide only part of the oscillometric envelope data. Using the method as described around FIG. 6, the envelope data from the two processing techniques can be combined to provide a complete oscillometric envelope from which blood pressure can be subsequently estimated.

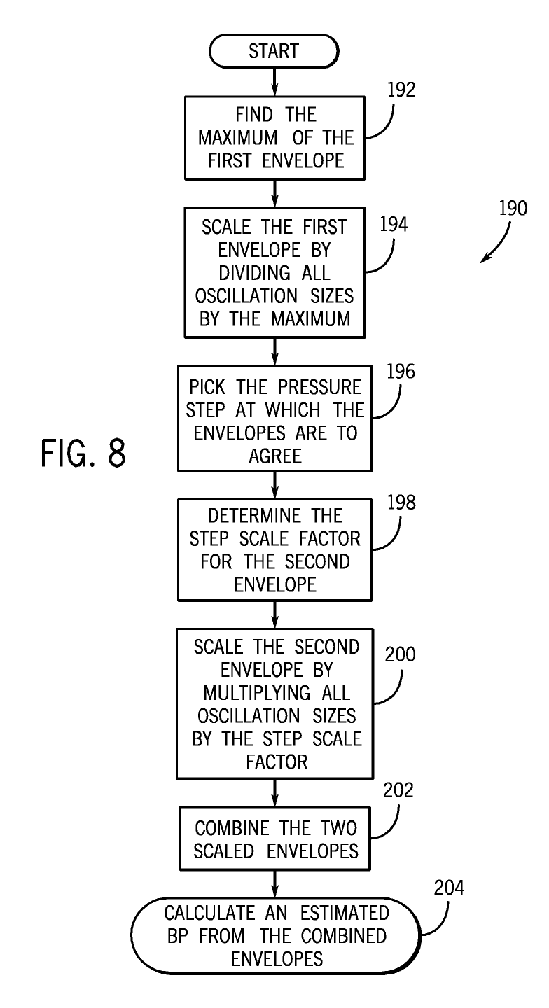

Referring back to FIG. 4, the step 152 of combining the first and second set of envelope points 154 can be carried out utilizing other types of combining techniques, other than that shown in FIG. 7. One additional type of combining technique 190 is shown in FIG. 8. The combining technique 190 can be utilized on the results of the first and second processing techniques, as shown in FIGS. 3a-3b, 51-5b and 6a-6b.

In the combining technique 190 shown in FIG. 8, the combining technique 190 initially finds the maximum of the first set of envelope points, as shown by step 192. In the dataset shown in FIG. 3a, the maximum 140 of the first set of envelope points 132 is selected.

Once the maximum 140 has been located, the envelope points 136 are divided by the maximum to scale each of the envelope points 136 to a value between 0 and 1, as shown by step 194. Since oscillometry is a ratio technique based on the relative size of the envelope, this normalization process applied to the first set of envelope points is not absolutely necessary but is included here for clarity and consistency in envelope combination. However, one additional advantage of this scaling is that knowing the precise pulse amplitude relative to the maximum could be used in deciding which particular step to use for calculating the scale factors when more than one choice is available.

After the first set of envelope points have been scaled, as illustrated by the reference numeral 160 in FIG. 3c, the second combining technique 190 selects a pressure step at which the first and second oscillometric envelopes shown in FIGS. 3a and 3b are to agree, as illustrated by step 196. As an example, in the data values shown in FIGS. 3a and 3b, both the first set of envelope points 132 and the second set of envelope points 146 include an oscillation amplitude at approximately 85 mmHg. Since both datasets include an amplitude near 85 mmHg, this pressure step is selected in step 196.

Once the pressure step has been selected, the second combining technique 190 calculates a step scale factor for the second envelope, as illustrated in step 198. Specifically, the step scale factor is determined by setting the scaled envelope point at the selected pressure step of the second set of envelope points to be the same as the scaled value of the envelope point 136 at the same pressure step in the first set of envelope points. As an example, if the scaled envelope point from the first set of envelope points 132 at the selected pressure step of 85 mmHg is 0.82, the envelope point 148 in the second set of envelope points 146 of the same pressure step revised to have a scaled value of 0.82.

Once the envelope point 148 for the selected pressure step of the second set of envelope points 146 is scaled to the same value as the envelope point 136 for the selected pressure step of the first set of envelope points, the remaining envelope points 148 of the second set of envelope points are scaled utilizing the same scaling factor, as illustrated in step 200. Therefore, the second set of envelope points 146 are scaled based upon the first set of envelope points 132. Once the two sets of envelope points 132, 146 are scaled as described, the two sets of envelope points are combined in step 202 to create the combined oscillometric data values, similar to the combinations as shown in FIGS. 3c, 5c and 6c. Based upon the combined data values, the system estimates a blood pressure in step 204.

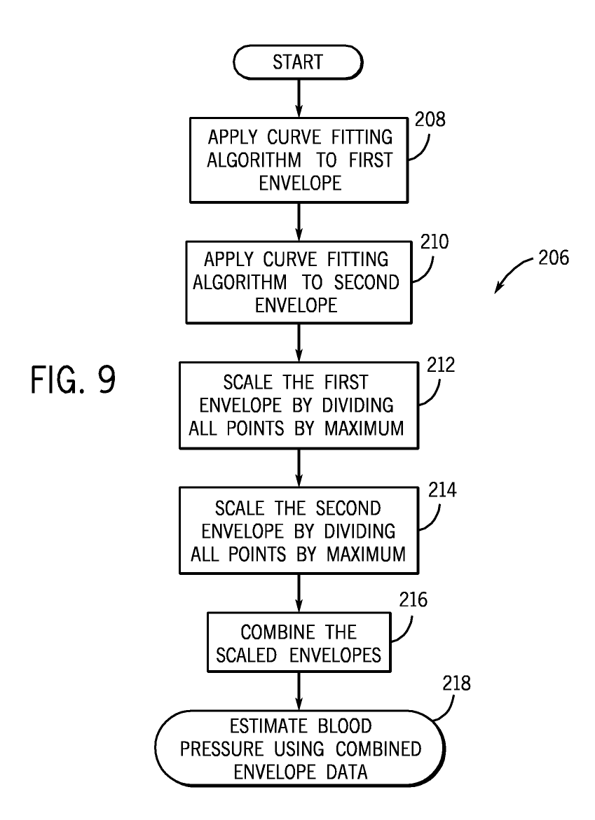

FIG. 9 illustrates yet another combining technique 206 that can be utilized in step 152 of FIG. 4 to combine the first and second sets of oscillometric envelope points. In the combining technique 206 shown in FIG. 9, the first step 208 in the process is to apply a curve fitting algorithm to the first set of envelope points 132 shown in FIG. 3a. The curve fitting algorithm is a standard algorithm utilized to generate an oscillometric envelope based upon the series of oscillometric data points.

After the curve fitting algorithm has been applied to the first set of envelope points, the same curve fitting algorithm is applied to the set of envelope points 146, as illustrated by step 210. The curve fitting algorithm utilized in step 210 is the same curve fitting algorithm utilized in step 208.

Once the curve fitting algorithm has been applied to the first and second set of envelope points, the combining technique 206 scales the first and second envelopes by dividing each of the envelope points by the maximum amplitude of the curve fit for each set, as shown in steps 212 and 214. Scaling of both of the first and second set of data points after the curve fitting algorithm using the curve fit maximums results in each of the envelope points having a value between 0 and 1.

After the envelope points have been scaled, the first and second set envelope points are combined in step 216. Since the first set of envelope points and the second set of envelope points are in the range of 0 to 1, the combined data points can be utilized in step 218 to estimate the blood pressure for the patient.

Although three different combining techniques 154, 190 and 206 are shown in the present disclosure, it should be understood that various other combining techniques could be utilized while operating within the scope of the present disclosure. In each case, the system and method utilizes multiple processing techniques to generate a set of envelope points. The two different sets of envelope points are combined utilizing one of the combining techniques described such that the envelope data points can be combined to generate a single blood pressure estimate.

Finally, it is some times necessary to combine the various sets of envelope points in such a way that the data from a particular set is weighted differently as the combination process proceeds to give one set of data more influence in determining the blood pressure estimates. After scaling, one way to easily accomplish this is to include the more important envelope data more than once in the final combined set. This could apply to data points within envelope sets or the entire envelope sets. The final combined set could then be used in a curve fitting procedure to estimate blood pressure values.

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

What is claimed is:

1. A method of determining the blood pressure of a patient using a non-invasive blood pressure (NIBP) monitor having an inflatable and deflatable blood pressure cuff and a pressure transducer, the method comprising the steps of:
obtaining a plurality of oscillometric waveform sample values from the pressure transducer at a plurality of pressure steps;
processing the oscillometric waveform sample values using a first processing technique to determine a plurality of first envelope points;
processing the oscillometric waveform sample values using a second processing technique to determine a plurality of second envelope points;
scaling at least the plurality of second envelope points;
combining the first envelope points and the scaled second envelope points; and
calculating the blood pressure from the combination of the plurality of first envelope points and the plurality of scaled second envelope points.

2. The method of claim 1 further comprising the steps of:
scaling the plurality of first envelope points;
combining the plurality of scaled first envelope points and the plurality of scaled second envelope points; and
calculating the blood pressure from the combination of the plurality of scaled first and second envelope points.

3. The method of claim 2 wherein the step of scaling the first and second data points comprises:
determining the first envelope point with the maximum amplitude;
dividing each of the plurality of first envelope points by the maximum amplitude to create the plurality of scaled first envelope points;
determining the second envelope point with the maximum amplitude; and
dividing each of the plurality of second envelope points by the maximum amplitude to create the plurality of scaled second envelope points.

4. The method of claim 1 wherein the plurality of second envelope points are scaled based upon the plurality of first envelope points.

5. The method of claim 4 further comprising the steps of:
selecting a scaling pressure step;
determining the amplitude of a first envelope point at the scaling pressure step;
determining the amplitude of a second envelope point at the scaling pressure step;
determining a scaling factor required to modify the amplitude of the second envelope point at the scaling pressure step to equal the amplitude of the first envelope point at the scaling pressure step; and
applying the scaling factor to each of the plurality of second envelope points.

6. The method of claim 1 wherein the first and second processing techniques are different from each other.

7. The method of claim 2 further comprising the steps of:
applying a curve fitting technique to the plurality of first envelope points;
applying the curve fitting technique to the plurality of second envelope points;
identifying a first envelope amplitude after application of the curve fitting technique;
dividing each of the plurality of first envelope points after application of the curve fitting technique by the first envelope amplitude to create the plurality of scaled first envelope points;
identifying a second envelope amplitude after application of the curve fitting technique; and
dividing each of the plurality of second envelope points after application of the curve fitting technique by the second envelope amplitude to create the plurality of scaled second envelope points.

8. The method of claim 5 wherein the step of selecting the scaling pressure step includes determining a pressure step that includes both a first envelope point and a second envelope point.

9. A method of determining the blood pressure of a patient, the method comprising the steps of:
positioning a blood pressure cuff on the patient, the blood pressure cuff having a pressure transducer;
inflating the blood pressure cuff to a target inflation pressure;
deflating the blood pressure cuff in a series of pressure steps;
obtaining a plurality of oscillometric waveform sample values from the pressure transducer at the series of pressure steps;
processing the oscillometric waveform sample values using a first processing technique to determine a plurality of first envelope points;
processing the oscillometric waveform sample values using a second processing technique to determine a plurality of second envelope points;
combining the plurality of first envelope points and the plurality of second envelope points; and
calculating the blood pressure from the combination of the first and second envelope points.

10. The method of claim 9 wherein the first processing technique and the second processing technique are different from each other.

11. The method of claim 9 further comprising the steps of:
scaling the plurality of second envelope points; and
combining the plurality of first envelope points and the plurality of scaled second envelope points.

12. The method of claim 11 wherein the second envelope points are scaled based upon the plurality of first envelope points.

13. The method of claim 9 further comprising the steps of:
determining the maximum amplitude of the plurality of first envelope points;
dividing each of the plurality of first envelope points by the maximum amplitude of the first envelope points to create a plurality of scaled first envelope points;
determining the maximum amplitude of the second envelope points;
dividing each of the plurality of second envelope points by the maximum amplitude of the second envelope points to create a plurality of scaled second envelope points; and
combining the plurality of scaled first envelope points and the plurality of scaled second envelope points; and
calculating the blood pressure from the combination of the plurality of first scaled envelope points and the plurality of scaled second envelope points.

14. The method of claim 9 further comprising the steps of:
selecting a scaling pressure step;
determining the amplitude of a first envelope point at the scaling pressure step;
determining the amplitude of a second envelope point at the scaling pressure step;
determining a scaling factor required to equate the amplitude of the second envelope point at the scaling pressure step to the amplitude of the first envelope point at the scaling pressure step; and
applying the scaling factor to each of the plurality of second envelope points, wherein the plurality of scaled envelope points are combined with the plurality of first envelope points.

15. A system for determining the blood pressure of a patient comprising:
a blood pressure cuff positionable on the patient, the blood pressure cuff including a pressure transducer, wherein the pressure transducer is used to generate a plurality of oscillometric waveform sample values at a series of pressure steps during the operation of controlling the pressure in the blood pressure cuff;
a first processing means that receives the plurality of oscillometric waveform sample values and generates a plurality of first envelope points using a first processing technique;
a second processing means that receives the oscillometric waveform sample values and generates a plurality of second envelope points utilizing a second processing technique;
means for combining the plurality of first envelope points and the plurality of second envelope points; and
means for calculating the blood pressure from the combination of the first envelope points and the second envelope points.

16. The system of claim 15 wherein the first processing technique and the second processing technique are different from each other.

17. The system of claim 15 wherein the means for combining the plurality of first envelope points and the plurality of second envelope points scales at least the plurality of second envelope points prior to combining the second envelope points with the first envelope points.

18. The system of claim 15 wherein the means for combining scales both the first envelope points and the second envelope points prior to combining the plurality of first envelope points and the plurality of second envelope points.

19. The system of claim 18 wherein the means for combining scales the plurality of second envelope points based upon the plurality of first envelope points.

20. The system of claim 15 wherein the means for processing the oscillometric data values to generate the plurality of first and second envelope points and the means for combining the plurality of first envelope points and the plurality of second envelope points are a common processor.
